(19) United States
(12) Plant Patent Application Publication
(10) Pub. No.: US 2002/0138887 P1
(43) Pub. Date: Sep. 26, 2002

(54) Gladiolus hybrid plant 'Palampur Princess'

(76) Inventors: Devashish Mukherjee, Palampur (IN); Devendra Dhayani, Palampur (IN); Jaichand Rana, Simla (IN)

Correspondence Address: BURNS DOANE SWECKER & MATHIS L L P, POST OFFICE BOX 1404, ALEXANDRIA, VA, 22313-1404, US

(21) Appl. No.: 09/813,829

(22) Filed: Mar. 22, 2001

(51) Int. Cl. A01H 5/00

(52) U.S. Cl. PLT/301

(57) ABSTRACT

The invention relates to a novel and distinct hybrid plant named ‘Palampur Princess’ (Gladiolus sp) characterized by its attractive Dutch Vermilion colored flowers with Dresden yellow color on the center of the lip petals.

### FIELD OF THE INVENTION

The present invention relates to a novel hybrid plant named “Palampur Princess” and belonging to the family Iridaceae. The novel plant being a hybrid, has been developed in a breeding programme. The novel plant “Palampur Princess” is propagated vegetatively by corms and hence can be maintained as a stable genotype. The plant of the invention is an ornamental plant widely cultivated for beautiful flowers which are of commercial and export value.

### BACKGROUND OF THE INVENTION

The present invention relates to a new and distinct variety of gladiolus, a member of the Gladiolus genus. The novel plant is the hybrid between the gladiolus plants Bonfire and Eurovision (Hybrid varieties).

Gladiolus is one of the important cut flowers throughout the world. The commercial cultivation is wide spread in temperate, tropical and subtropical climates. The demand of new varieties with better colour, quality flowers, and planting materials is always existing in the floriculture trade.

The modern garden cultivators gladiolus come from diverse genetic parentages. It has cumulative heterozygosity for many characters inherent with complex genetic constitution. In gladiolus, diverse parents are crossed together and the cultivars and the species that differ widely in chromosome numbers are also cross-fertile. In the present invention, the desirable strains obtained in F₁ generation were perpetuated vegetatively without being segregated in the following generations, so that the cultivars which are available today may be F₂, F₃ to F₈ or so of a particular cross further blended with some extra parents at nearly every generation. Thus they are not allowed to segregate freely in further generations because it is desirable to grow the plants asexually. Because of this reason, now the available modem cultivars have become so complex that the offspring obtained by crossing them, even two seedlings, do not appear similar [(Misra, 1975) Gladiolus Br. Assn. Newsletter, No.12, pp.2-5].

The Applicants collected germplasm of different cultivars and hybrid varieties of gladiolus from National Botanical Research Institute Lucknow, India as per the list of gladiolus cultivars grown in India and described in the bulletin of ‘gladiolus’ Economic Botany Information Service by Sharma et al. published by the Director National Botanical Research Institute Lucknow, 1988. Germplasm of gladiolus was also collected from Netherlands in 1991 and various nurseries of Kalimpong, Darjeeling, West Bengal, India. The record of the collected germplasm of gladiolus was maintained in the accession register of the Floriculture Division of the Institute of Himalayan Bioresource Technology (IHBT), Palampur, India.

The applicants initiated a breeding program to develop better types of gladiolus hybrids suitable to wide range of climatic conditions, and having wide range of characteristics such as better colour, increased number of florets and spike length as per the international standards, better yield of corms and cormels, tolerant to the common diseases etc. The collected germplasm of gladiolus was planted in the experimental field of IHBT for their propagation and multiplication. In this breeding programme conventional breeding method (hybridization) was used. More than 100 cross combinations were made by using distinct varieties such as Oscar, Jester, Snow Princess, Eurovision, Ballerina, King Liar, Cherry Blossom, Her Majesty, Green woodpecker, Friendship, Vink's Glory, Aldebaran, Red Beauty, Top Brass, Copper King, Bonfire, White Goddess, Sunny Boy, Tropic Sea and Friendship Pink etc.

Color description of some of the parentage as described in NAGC Bulletin.

Oscar—Turkey red, throat blotched sulphur yellow.

Green Woodpecker—Pea Green, throat blotched pea green spotted ruby red.

Eurovision—Signal red, throat streaked pea green.

Friendship Pink—Dawn Pink, throat blotched pea green having splashes ruby red.

Aldebaran—Straw Yellow and throat bloched signal red.

As the aim is the production of seed of known parentage, emasculation in first three flowers in a selected spike is done before the opening of the flowers and stigma becomes receptive. Anthers are removed carefully from each flower. Emasculated flowers were covered with butter paper bags used for breeding purposes. Pollination was done in the emasculated flowers next day morning with in 24-30 hours with the pollens of the desired parents in the month of April-May 1991. The seeds were collected from mature pods in the month of August-September and were sown in beds under open field conditions and covered with dry grasses for moisture preservation in December 1991. The resultant seedlings were space planted in the field at Palampur in March-April 1992.

Many seedlings came out from a single cross combination. These plants were critically evaluated and tagged as per the desired colour combinations, growth and flowering parameters. The corn and cormels of the selected hybrid plants were replanted continuously four years in the filed for further evaluation and multiplications. Based on the superior performance for attractive colour combination, compactness of flower spike, number of flowers per spike, length of flower spike, Number of corm and cormels per plant evaluation and selection of superior quality hybrids were made. Thus, the breeding program involved hybridisation of commonly available gladiolus

plants. In other words, the hybrids were developed by crossing parental genotypes involving sexual hybridization in the breeding programme.

The program yielded a number of hybrid plants out of which one genotype namely IHBT-GH-272 was selected and christened as ‘Palampur Princess”. This plant was found to have new colour, flower size, number of florets per spikes, length of flower spikes, better yield of corms and cormels and less prone to common diseases. Growing the plant on a commercial scale offers the horticulturists an improved and new variety, which can be commercially cultivated.

### DETAILED DESCRIPTION OF THE INVENTION

Thus, the invention provides a new genotype christened as “Palampur Princess”. This plant has been developed through planned breeding experiments conducted at Institute of Himalayan Resources, (IHBT) Palampur, Himachal Pradesh, India with defined aim to develop superior gladiolus genotypes. For this purpose, gladiolus varieties were collected from different sources and grown in the fields at Palampur, India for facilitating breeding program. The emasculation and pollination in different varieties were carried out during the months of April-May 1991. The seeds were collected in July-August 1991 and sown in beds under open field conditions and covered with dry grasses in December 1991. The resultant seedlings were space planted in the field at Palampur in March-April 1992. The corm and cormels of survived hybrid plants were replanted continuously four years for screening and multiplication.

Based on the superior performance for attractive colour combination, compactness of flower spikes, number of flowers per spikes, length of flower spikes, number of flowers remains open at a time, number of corms and cormel production per plant, the plant of this invention (IHBT-GH-272) was selected for further observation and evaluation.

Considering the superior characteristics like excellent colour, number of flowers, compactness of flower spikes, plant height, ruffled-ness of flower petals, regeneration potential and freedom from common diseases, it was asexually reproduced through corms and cormels to maintain purity.

The selected hybrid IHBT-GH-272 was christened as “Palampur Princess” and grown at row distance of 1 feet and plant to plant 6 inches for four consecutive years to study its growth and flowering performance and multiplication. Data were recorded on randomly selected twenty plants every year. The hybrid IHBT-GH-272 maintained uniformity in its growth and flowering performance.

### EVIDENCE OF UNIFORMITY AND STABILITY

The hybrid IHBT-GH-272 has remained stable and uniform for its morphological characters and showed consistency in performance for various growth and flowering parameters during its evaluation and vegetative multiplication since 1992. Throughout the evaluation period of IHBT-GH-272 no variants were found from the normal population.

The genotype IHBT-GH-272 possesses decorative type flowers of Dutch Vermillion (RHS-40A) with Dresden yellow (RHS-5D) colour on center of the lip petals. The flower petals are slightly ruffled which is quite clear from FIG. 1.

The genotype IHBT-GH-272 is distinct in regeneration potential.

### BRIEF DESCRIPTION OF THE ACCOMPANYING PHOTOGRAPH

FIG. 1 is a photograph of field grown flower spike of IHBT-GH-272 (‘Palampur Princess’) depicting decorative type flowers of Dutch Vermillion (RHS-40A) with Dresden yellow (RHS-5D) colour on center of the lip petals.

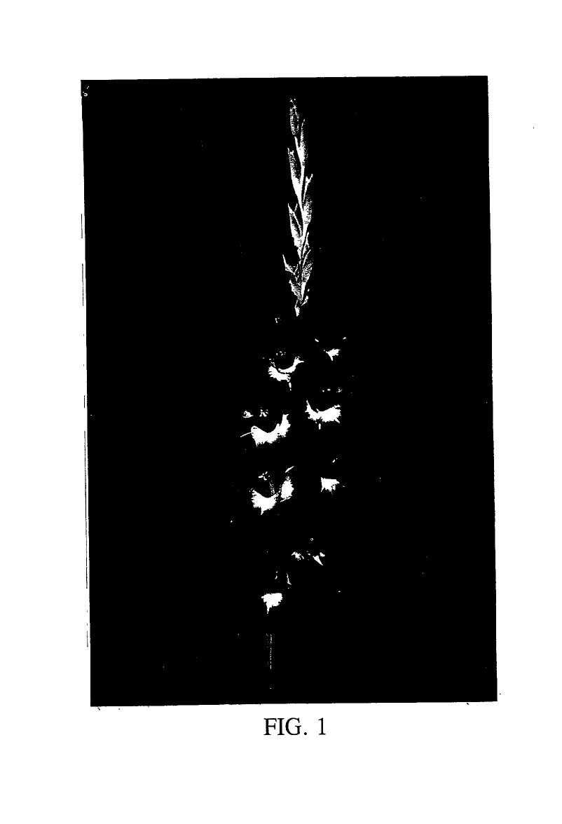

The plant of invention “Palampur Princess” is thus a new and distinct hybrid plant, having the following combination of characters:

a) colour flowers of Dutch Vermilion (RHS-40A) with dresden yellow color (RHS-5D) on the center of the lip petals;

b) ruffled petals

c) Flowering period: about 84 days.

d) average number of flower spikes/ plant is 1.79

e) average length of flower spikes is 95.9 cm.

f) average number of flowers per spikes is 16.

### OBJECTIVE DESCRIPTION OF THE GENOTYPE “PALAMPUR PRINCESS”

The following is an objective description of the new variety.

1. Genus:Gladiolus

2. Species:Hybrid sp.

3. Family:Iridacae

4. Common name:Sward lily/gladiolus.

5. Plant height:114.13 cm

6. Growth habit:Erect, uniform.

7. Stem diameter:1.12 cm

8. Number of leaves/plant:7.32

9. Height of leaves:54.03 cm

10. First flowering:84 days

11. Type of flowers - Decorative

12. Number of spikes/plant: 1.79

13. Length of the flower spike: 95.9 cm

14. Number of flowers/spike: 16

15. Flower colour: Dutch Vermillion (RHS-40A) with Dresden yellow (RHS-5D) colour on center of the lip petals.

16. Type of petals: Petals slightly ruffled

17. Number of flowers remains open at a time: 8.0

18. Longevity of the 1st flower: 3.15 days

19. Diameter of 1st flower: 9.79 cm

20. Longevity of the spike: 9.61 days

21. Number of corms/plant: 1.77

22. Diameter of corms: 5.04 cm

23. Number of cormels/plant: 146

(a) Type: Decorative

(b) Days to flower: 84 days

1. A novel and distinct gladiolus plant christened as “Palampur Princess” characterized by the following combination of characteristics:
a) having flowers of Dutch vermillion color with dresden yellow color on the center of the lip petals;
b) having at least 1.79 spikes per plant;
c) length of flower spike is 95.9 cm;
d) number of flowers per spike being 16;
and substantially as herein described and illustrated.
